(19) United States
(12) Patent Application Publication
(10) Pub. No.: US 2006/0034910 A1
(43) Pub. Date: Feb. 16, 2006

(54) Pharmaceutical composition for extended release of phenytoin sodium

(76) Inventors: Sanjay Patel, Rockville, MD (US); Gour Mukherji, Gurgaon (IN); Jayadev Patil, Gulbarga (IN); Sidharth Mate, Nagpur (IN)

Correspondence Address: Dr. O. M. (Sam) Zaghmout, 8509 Kernon Ct, Lorton, VA, 22079, US

(21) Appl. No.: 11/199,169

(22) Filed: Aug. 9, 2005

Related U.S. Application Data
(60) Provisional application No. 60/600,114, filed on Aug. 9, 2004.

(51) Int. Cl. A61K 9/48; A61K 9/26

(52) U.S. Cl. 424/451; 424/469

(57) ABSTRACT

The present invention provides an oral extended release solid pharmaceutical composition of phenytoin sodium or its pharmaceutically acceptable derivative thereof and the process of manufacturing the same the said extended release oral solid pharmaceutical composition comprising of at least one suitable pharmaceutically acceptable excipient alongwith phenytoin sodium. The said extended release oral solid pharmaceutical composition is manufactured by blending phenytoin sodium with at least one suitable pharmaceutically acceptable excipient and granulating the blend optionally using at least one suitable binder dissolved in an organic solvent or mixture of solvents.

CROSS REFERENCE TO RELATED APPLICATION

This application claims the benefit of U.S. Provisional Application No. 60/600,114 filed on Aug. 9, 2004. The entire disclosure of this prior application is hereby incorporated by reference.

### FEDERALLY SPONSORED RESEARCH

This invention has been created without the sponsorship or funding of any federally sponsored research or development program.

### SEQUENCE LISTING OR PROGRAM

Not Applicable.

### BACKGROUND OF THE INEVNTION—FIELD OF INVENTION

The present invention relates to a pharmaceutically acceptable oral formulation comprising antiepileptic agents, in particular phenytoin sodium and a process for preparing such a formulation.

### BACKGROUND OF INVENTION

Epilepsy refers to a disorder of brain function characterized by the periodic and unpredictable occurrence of seizures. Seizure refers to transient alteration of behavior due to the disordered, synchronous, and rhythmic firing of populations of brain neurons. i.e. A seizure is a change in sensation, awareness, or behavior brought about by a brief electrical disturbance in the brain. Seizures vary from a momentary disruption of the senses, to short periods of unconsciousness or staring spells, to convulsions.

Several drugs (called antiepileptic or anticonvulsant drugs) are prescribed to prevent seizures. Hydantoins are effective against all types of partial and tonic-clonic seizures. Phenytoin was first in the series of nonsedative antiepiletics available for the treatment of seizures. Other such agents for the acute treatment of epilepsy also include mephenytoin and ethotoin.

Phenytoin, 5,5-diphenyl-2,4-imidazolidinedione, is used mainly in the prophylactic management of tonic-clonic seizures and partial seizures with complex symptomatology. The drug is also effective in controlling autonomic seizures. The primary site of action appears to be the motor cortex where spread of seizure activity is inhibited. Possibly by promoting sodium efflux from neurons, phenytoin tends to stabilize the threshold against hyperexcitability caused by excessive stimulation or environmental changes capable of reducing membrane sodium gradient. This includes the reduction of posttetanic potentiation at synapses. Loss of posttetanic potentiation prevents cortical seizure foci from detonating adjacent cortical areas. Phenytoin reduces the maximal activity of brain stem centers responsible for the tonic phase of tonic-clonic (grand mal) seizures. Several formulations of phenytoin have been reported in the literature, many of which relate to formulations of phenytoin sodium in particular. For example, formulations relating to oral, parenteral, immediate release and extended or modified release compositions for phenytoin have been reported. Examples of patents describing such formulations are as follows.

U.S. Pat. No. 6,274,168 (Mylan Pharmaceuticals Inc.) relates to an extended release pharmaceutical composition comprising an admixture of phenytoin sodium and an erodible matrix, wherein the matrix comprises a pharmaceutically acceptable binders, diluents, or combination thereof. The composition constitutes a tablet in a capsule, wherein said tablet comprises a phenytoin species having a characteristic Raman shift in the erodible matrix, obtained after granulation with an aqueous solvent. It has been stated that the presence of an adequate amount of the aqueous solvent is important to the granulation process because the water is believed to dissolve the phenytoin sodium, regardless of its polymorphic form, so that upon drying and resolidification, the resulting mixture will contain a unique phenytoin species characterized by the Raman shift. Yet, the disadvantage of using water as solvent are many. When water is used as solvent for water-soluble drug like phenytoin sodium, the detection of granulation endpoint is unclear as when the end-point is reached, the drug starts dissolving and as a result, the mass may turn into overly wet material which dries slowly and forms hard aggregates, which tend to turn to powder during subsequent dry milling. Also, water may adversely affect drug stability, due to longer drying time and extended exposure to heat. A part of the phenytoin sodium may dissolve and recrystallize to a different polymorphic form, whereas the remaining portion may remain in the unchanged polymorphic form. This mixture of polymorphs may exhibit different dissolution characteristics and give rise to variable dissolution during the shelf-life of the product.

In a PCT Application (WO 02/092056 A1), Warner Lambert Company relates to a process of preparing a pharmaceutical formulation comprising the steps of (a) adding sodium phenytoin to a vessel of a blender, (b) adding at least one excipient to said vessel, (c) blending said excipient and said sodium phenytoin to form a blend, (d) compacting said blend to form a compact, and (e) milling said compact to form a granulation. The essence of this invention is a shorter process time compared to a wet granulation process as this process relates to dry compaction, followed by milling and compression.

U.S. Pat. No. 5,968,554 (Cascade Development, Inc.) relates to sustained release pharmaceutical preparations and to a method for making them. The novel drug delivery system described therein contains a core comprising the active, an enteral coating over the core comprising a pH dependent water-soluble polymer, a second coating of the active and thereafter a coating, which is soluble in gastric juices. The drug delivery system of the invention may be utilized with a wide variety of pharmaceutically active agents including phenytoin sodium, which have pH dependent solubilities to prepare sustained release compositions. Karakasa et al., Biol. Pharm. Bull., 1994; 17 (3): 432-436 in an article entitled “Sustained Release of Phenytoin Following the Oral Administration of Sodium Phenytoin/Ethyl cellulose Microcapsules in Human Subjects and Rabbits,” studied the release patterns of phenytoin sodium in combination with ethyl cellulose. The sodium phenytoin microcapsules were prepared by mixing 80% (by weight) of the sodium phenytoin in 10% (by weight) ethyl cellulose solution in ethyl acetate. The suspension was stirred and n-pentane was added dropwise until a phase separation occurred and the microcapsules were obtained. The article reports that following the oral administration of sodium phenytoin; the salt might be easily transferred into free phenytoin in the acidic fluids of the stomach. As free-phenytoin is practically insoluble in water, its absorption might be incomplete in the gastrointestinal tract. On the other hand, while passing through the stomach, the volume of water penetrating into the ethylcellulose microcapsules might be minimal. Thus, most of the sodium phenytoin in the microcapsules might not be converted into free phenytoin.

In a PCT Application (WO 04/075826 A2), inventors of Ranbaxy Ltd. relates to oral extended release multiple unit dosage forms of phenytoin sodium in which individual units comprising phenyoin sodium are coated with one or more film forming polymers. The said film forming polymers are selected from cellulose derivatives, vinyl polymers and copolymers, acrylic polymers and copolymers and biodegradable polymers. The multiple unit formulation may be a capsule or a tablet that disintegrates in the stomach to give individual units.

It will be appreciated from the prior art discussed above that many different formulations for immediate release, extended release or modified release phenytoin have been described in the prior art.

Despite the availability of different technologies for phenytoin formulations, there is a clinical need for better preparations that are simple, stable and can confer an unswerving dissolution profile and are manufactured by an expedient manufacturing process of wet granulation using organic solvents. As literature reports that wet granulation is a preferred route of granulation as it is known to improve flow, compressibility, bioavailability, and homogeneity of the blends, electrostatic properties of powders, and stability of dosage forms.

To this end, the present invention involves an extended release compositions of phenytoin sodium or its pharmaceutically acceptable derivative thereof comprising phenytoin sodium alongwith at least one suitable excipient, and the said composition is manufactured by granulating the blend of phenytoin sodium and the said excipient optionally using at least one suitable binder dissolved in organic solvent or mixture of solvents so as to prevent the conversion of phenytoin sodium into any other polymorphic form.

### SUMMARY OF THE INVENTION

The present invention provides an oral extended release solid pharmaceutical composition of phenytoin sodium or its pharmaceutically acceptable derivative thereof and the process of manufacturing the same the said extended release oral solid pharmaceutical composition comprising of at least one suitable pharmaceutically acceptable excipient alongwith phenytoin sodium. The said extended release oral solid pharmaceutical composition is manufactured by blending phenytoin sodium with at least one suitable pharmaceutically acceptable excipient and granulating the blend optionally using at least one suitable binder dissolved in an organic solvent or mixture of solvents so as to retain the polymorphic form of phenytoin sodium.

Accordingly, it is an object of the present invention to provide a pharmaceutical composition and a process of manufacturing the same for the time-specific delivery of antiepileptic drugs like phenytoin sodium or its pharmaceutically acceptable derivative thereof.

The present invention, therefore also provides a pharmaceutical formulation comprising at least one antiepileptic pharmaceutically active agent alongwith at least one or more suitable pharmaceutical excipient.

Another aspect of the present invention is an extended release, solid pharmaceutical composition adapted for oral administration. This composition comprises of at least one antiepileptic pharmaceutically active agent, particularly, phenytoin sodium or its pharmaceutically acceptable derivative thereof together with at least one or more suitable pharmaceutically acceptable carrier or excipient thereof.

Yet another aspect of the present invention is the process of manufacturing the granules of oral solid extended release pharmaceutical composition. In other words, the invention provides an extended release, solid pharmaceutical composition comprising at least one antiepileptic pharmaceutically active agent, particularly, phenytoin sodium or its pharmaceutically acceptable derivative thereof together with at least one suitable pharmaceutically acceptable carrier or excipient blended suitably and granulated optionally with suitable binder dissolved in organic solvent or mixture of solvents thereof so as to retain the polymorphic form of phenytoin sodium.

The present formulation process provides obvious benefits being simple and fast operational process for manufacturing said oral solid extended release pharmaceutical composition.

### BRIEF DESCRIPTION OF THE DRAWINGS

FIG. 1 illustrates the comparative dissolution profiles of Phenytoin capsule formulation (Example 1) versus the Dilantin Kapseals® 100 mg in water.

FIG. 2 illustrates the comparative dissolution profiles of Phenytoin capsule formulation (Example 2) versus the Dilantin Kapseals® 100 mg in water.

FIG. 3 illustrates the comparative dissolution profiles of Phenytoin capsule formulation (Example 2) versus the Dilantin Kapseals® 100 mg in 0.001 N Hydrochloric acid.

FIG. 4 illustrates the comparative dissolution profiles of Phenytoin capsule formulation (Example 2) versus the Dilantin Kapseals® 100 mg in pH 6.8 phosphate buffer.

### DETAILED DESCRIPTION OF THE INVENTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

The information provided below is not admitted to be prior art to the present invention, but is provided solely to assist the understanding of the reader.

The details of one or more embodiments of the invention are set forth in the accompanying drawings and the description below. Other features, objects, and advantages of the invention will be apparent from the description and drawings, and from the claims.

The extended release pharmaceutical composition of the present invention comprises therapeutically effective amount of phenytoin sodium or its pharmaceutically acceptable derivatives thereof and at least one suitable excipient blended together and granulated optionally with a binder suitably dissolved in organic solvent or mixture of solvents.

The term “pharmaceutically acceptable derivative” means various pharmaceutical equivalent isomers, enantiomers, complexes, salts, hydrates, polymorphs, esters etc of phenytoin

The term composition includes but not limited solutions and/or suspensions, dispersions, concentrations, ready mix, powders, granules, granules comprising phenytoin sodium or its pharmaceutically acceptable derivative thereof and at least one or more pharmaceutically acceptable excipient.

The term “therapeutically effective amount” means an amount of the drug which is capable of eliciting a physiological response in a human patient. More particularly, the term “therapeutically effective amount” means the amount of drug, which is capable of treating epilepsy and other related disorders.

The said medicament according to the present invention comprises a formulation substantially as herein described, and in particular a capsule or a tablet or granules filled in capsule formulation, typically an extended release capsule formulation substantially as hereinafter further described.

Suitably, a formulation according to the present invention provides an extended release capsule dosage form comprising granules of phenytoin sodium and at least one suitable excipient in suitable amounts, blended together and granulated optionally with at least one binder suitably dissolved in organic solvent or mixture of solvents.

In a preferred embodiment of the present invention, an extended release formulation comprises pharmaceutically active agent together with suitable excipient. In particular, the present invention provides extended release capsule or granules comprising an extended release source of at least one antiepileptic agent. A preferred active for use in capsule according to present invention is phenytoin sodium. As such in a formulation according to the present invention, release of phenytoin sodium after oral administration can be extended over minimum period of at least two hours. This extended release is achieved by the formulation substantially as hereinbefore described.

A capsule or granules or pellets (to be filled in capsule) according to the present invention comprises at least one or more suitable excipients.

The above materials are combined with phenytoin sodium or its pharmaceutically acceptable derivative thereof in specific concentration, to achieve the extended release characteristics of the present invention.

In general, the present invention provides a process for the manufacture of a pharmaceutical product. The process comprises blending of phenytoin sodium or its pharmaceutically acceptable derivative thereof in a suitable blender along with at least one or more suitable pharmaceutical excipient. The resultant blend is granulated optionally with binder solution prepared by dissolving at least one binder or mixture of binders in organic solvent or mixture of solvents. The granules are sized, lubricated and filled in capsules. The advantage of the process is it's fast operation and it also gives accurate granulation endpoint resulting in consistent eminence product. Another desired feature of the invention is the use of non-aqueous solvent that does not permit the dissolution of phenytoin sodium in the solvent and hence, there is no possibility for conversion of the initial drug into any other polymorphic form. This is further characterized by typical Raman shifts at 1600 and 1700 cm-1.

A Raman spectrum typically contains a large amount of information as sharp, well-resolved spectral bands. The band positions, intensities, and shapes provide an interpretable and fairly unique fingerprint for qualitative analysis. Raman spectra are measured by passing a laser beam through the sample and observing the scattered light either perpendicular to the incident beam or through backscatter detection. The scattered light is analyzed at high resolution by a monochromator and ultimately detected by a suitable device. Raman spectra are generally not complicated by contributions from adventitious moisture, owing to the weak scattering nature of the water molecule.

Raman spectroscopy is used primarily as means for the differentiation of polymorphs or solvates. It is also useful for determining the identity of organic and inorganic species in solution, as the Raman transitions for these species are more characteristics than for IR, where the transitions are much more affected by the other species present in the solution.

Raman is also very useful for measuring symmetric vibrations from C-C backbone of polymers.

The present invention provides the process for manufacturing extended release formulation of phenytoin sodium or its pharmaceutically acceptable derivatives thereof. Phenytoin sodium is water-soluble and is available in more than one polymorphic form. The characteristic Raman shifts for phenytoin sodium displays two shifts between 1600 and 1700 cm-1. The same is retained even after the granulation with nonaqueous solvents; probably as the non-aqueous solvents do not permit the dissolution of phenytoin sodium in the solvent and hence, there is no possibility for conversion of the initial drug into any other polymorphic form.

Suitable excipients employed in a pharmaceutical formulation according to the present invention, may include commonly used pharmaceutical excipients such as microcrystalline cellulose, dibasic calcium phosphate, hydroxypropylmethyl cellulose, powdered cellulose, carboxymethyl cellulose, hydroxyethylcellulose, and other cellulose derivatives magnesium oxide, calcium carbonate, magnesium aluminum silicate and other mineral bases.

The present invention may further comprise of suitable binders. The artisan can select appropriate binder soluble in organic solvents to granulate the blend. These may include hyroxypropylcellulose, hydroxyethyl cellulose, hydroxypropylmethyl cellulose, carboxymethyl cellulose, ethyl cellulose, methylcellulose, povidone, gum and gum derivatives, gelatin, polymethacrylates and its derivatives thereof.

An extended release formulation according to the present invention may also include pharmaceutically acceptable lubricants and glidants. The artisan can select appropriate lubricants and glidants in order to get good flow to aid in filling of capsules.

Examples of suitable lubricants and glidants include magnesium stearate, calcium stearate, zinc stearate, glyceryl behenate, light mineral oil, polyethylene glycol, sodium stearyl fumarate, stearic acid, talc, hydrogenated vegetable oil, calcium silicate, magnesium silicate and colloidal silicon dioxide. The most preferred pharmaceutical lubricant and glidants are talc, magnesium stearate and silicon dioxide.

The present invention further comprises a process of preparing a pharmaceutical product, or a pharmaceutical formulation, or a medicament substantially as hereinbefore described. Suitably such a process comprises providing at least one antiepileptic drug together with at least one excipient blended together and granulated optionally with a suitable binder dissolved in organic solvent or mixture of the solvents so as to prevent the conversion of initial drug into any other polymorphic form, and the said process thus yields an extended release pharmaceutical formulation, typically in capsule or granules (to be filled in capsule) form.

The present invention will now be further illustrated by the following example, which does not limit the scope of the invention in any way. Further different strengths of the formulation may be achieved by proportionately using a dose weight scale up or scale down formula. The concentrations of the excipients may also be varied or modified to achieve the desired dissolution profile by a skilled artisan.

### EXAMPLES

### Example 1

Extended release tablets were prepared using the following materials in the stated quantities:

| Serial | | Quantity | Quantity |
|---|---|---|---|
| No. | Ingredients | (mg/Capsule) | (% w/w) |
| 1. | Phenytoin sodium | 100.0 | 55.56 |
| 2. | Magnesium oxide | 10.0 | 5.56 |
| 3. | Hydroxypropyl methylcellulose | 51.0 | 28.33 |
| | (Methocel E-6 Premium LV) | | |
| 4. | Hydroxypropyl methylcellulose | 4.0 | 2.20 |
| | (6 cps) | | |
| 5. | Isopropyl alcohol | q.s. | — |
| 6. | Methylene chloride | q.s. | — |
| 7. | Colloidal silicon dioxide | 2.0 | 1.11 |
| 8. | Talc | 2.0 | 1.11 |
| 9. | Hydroxypropyl methylcellulose | 8.0 | 4.44 |
| | (Methocel E-15 Premium LV) | | |
| 10. | Magnesium stearate | 3.0 | 1.68 |
| | Total | 180.0 | 100.00 |

Procedure:

Blend 100.0 gm of phenytoin sodium, 10.0 gm of magnesium oxide and 51.0 gm of hydroxypropyl methylcellulose (Methocel E-6 premium LV). A binder solution of 4 gm of hydroxypropyl methylcellulose (6 cps) in a mixture of 100.0 gm of isopropyl alcohol and methylene chloride (50:50) was prepared. The above blend was granulated with the binder solution. The resulting granulation was dried, milled and blended with 2.0 gm of colloidal silicon dioxide, 2.0 gm of Talc, 8.0 gm of Methocel E-15 premium LV and 3.0 gm of magnesium stearate. The blended material was filled in size ‘3’ hard gelatin capsules by using a capsule-filling machine.

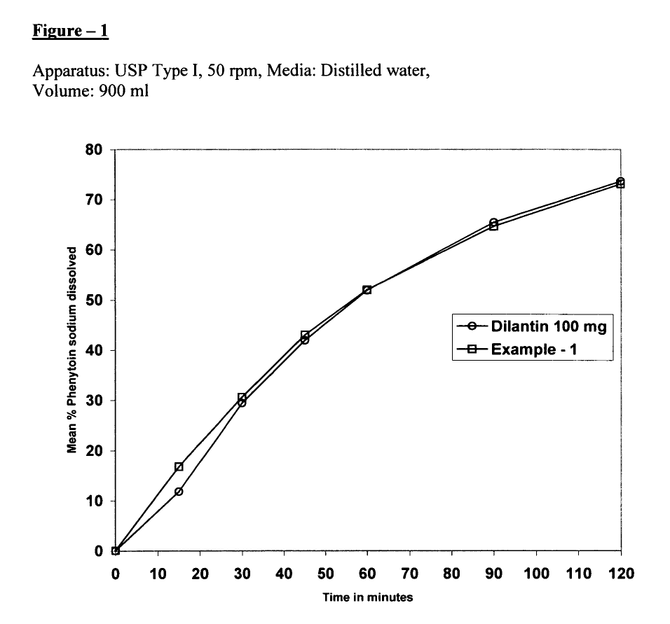

FIG. 1 shows the comparative dissolution profiles of the said invented capsule formulation and the reference formulation (Dilantin Kapseals 100 mg).

### Example 2

Extended release tablets were prepared using the following materials in the stated quantities:

| Serial | | Quantity | Quantity |
|---|---|---|---|
| No. | Ingredients | (mg/Capsule) | (% w/w) |
| 1. | Phenytoin sodium | 100.0 | 55.56 |
| 2. | Magnesium oxide | 10.0 | 5.56 |
| 3. | Hydroxypropyl methylcellulose | 55.0 | 30.56 |
| | (Methocel E-6 Premium LV) | | |
| 4. | Isopropyl alcohol | q.s. | — |
| 5. | Methylene chloride | q.s. | — |
| 6. | Colloidal silicon dioxide | 3.0 | 1.66 |
| 7. | Talc | 9.0 | 5.00 |
| 8. | Magnesium stearate | 3.0 | 1.66 |
| | Total | 180.0 | 100.00 |

Procedure:

Blend 100.0 gm of phenytoin sodium, 10.0 gm of magnesium oxide and 55.0 gm of hydroxypropyl methylcellulose (Methocel E-6 premium LV). A solvent mixture is prepared by using isopropyl alcohol and methylene chloride (70:30). The above blend was granulated with this solvent mixture. The resulting granulation was dried, milled and blended with 3.0 gm of colloidal silicon dioxide, 9.0 gm of Talc, and 3.0 gm of magnesium stearate. The blended material was filled in size ‘3’ hard gelatin capsules by using a capsule-filling machine.

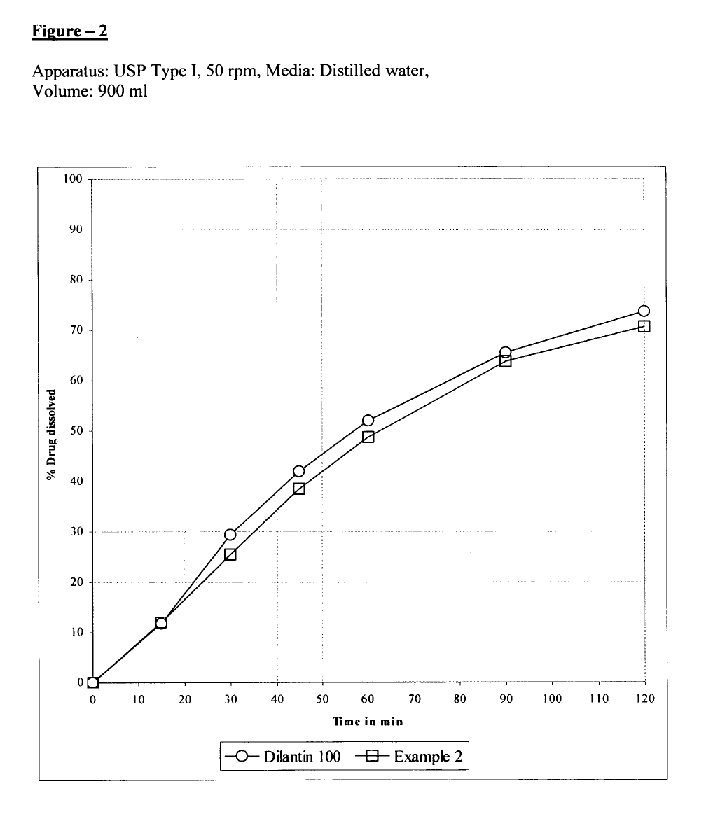

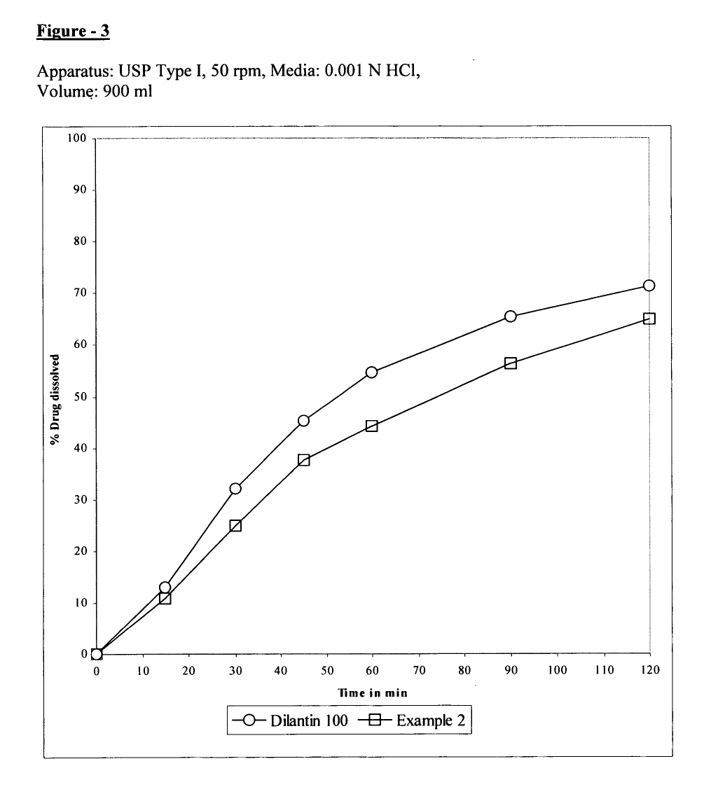

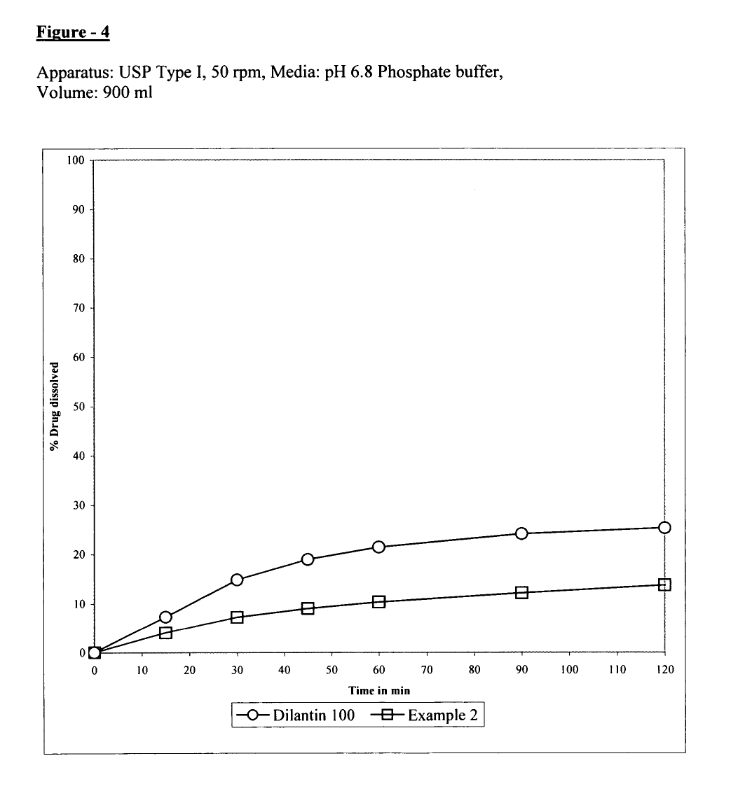

FIGS. 2, 3 and 4 shows the comparative dissolution profiles of the said invented capsule formulation and the reference formulation (Dilantin Kapseals 100 mg).

### Example 3

The bioequivalence of the pharmaceutical formulation of the present invention (Phenytoin sodium ER 30 mg and 100 mg capsules and that of the extended release capsules of phenytoin sodium available commercially (Dilantin Kapseals 100 mg) were studied. A, single-dose, open label, randomized comparative and two-way crossover pharmacokinetic study with a seven day washout period, was undertaken for the same.

Phenytoin sodium ER 100 mg was used as test product and Dilantin Kapseals® extended release 100 mg capsules was used as the reference product.

The pharmacokinetics assessment was based on the plasma levels of Phenytoin measured by blood sampling from healthy, adult, human subjects under fasting conditions.

Subjects received a single oral dose of Phenytoin sodium ER 100 mg capsules (test product) and a single dose oral dose of Dilantin Kapseals® extended release 100 mg capsules (reference product) with water at ambient temperature after the overnight fast for the study.

Table—I below displays both test/reference ratio and 90 % CI (Confidence Interval) for Cₘₐₓ and AUC of Phenytoin for the test and reference product.

TABLE I
Analyte: Phenytoin

| N = 18 (4 way | | Cₘₐₓ | AUCₗₐₛₜ | AUC_{inf} |
|---|---|---|---|---|
| crossover) | | (ng/ml) | (hr * ng/ml) | (hr * ng/ml) |
| Mean | Reference | 1452.70 | 42245.7 | 44930.5 |
| | Test | 1634.34 | 44133.2 | 46611.7 |

| | | | |
|---|---|---|---|
| T/R Ratio | 112.5 | 107.0 | 106.2 |
| CI (90%) | 103.8-122.0 | 99.9-114.6 | 99.4-113.4 |

Hence, it was observed that mean plasma phenytoin concentrations following single dose oral administration of test formulation were comparable to the mean plasma concentrations of Phenytoin following administration of reference Dilantin Kapseals® extended release formulation.

Therefore, oral Phenytoin ER formulation was found to be bioequivalent to the Reference Dilantin Kapseals® extended release capsule formulation.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks and other references (e.g., patents, journal articles) that contain definitions and methods and means for carrying out basic techniques, encompassed by the present invention.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A pharmaceutical formulation comprising:
A. A therapeutically effective amount of Phenytoin sodium or a pharmaceutically acceptable derivative thereof which does not form an erodible matrix and
B. One or more suitable pharmaceutically acceptable excipients,
wherein said formulation provides an extended or modified release of Phenytoin sodium or the pharmaceutically acceptable derivative thereof

2. An extended release composition of claim 1, comprising phenytoin sodium or a pharmaceutically acceptable derivative thereof and at least one suitable pharmaceutically acceptable excipient.

3. The pharmaceutical composition of claim 1, wherein the said pharmaceutically acceptable excipient is selected from the group consisting of microcrystalline cellulose, dibasic calcium phosphate, hydroxypropyl methyl cellulose, powdered cellulose, carboxymethyl cellulose, hydroxyethylcellulose and other cellulose derivatives, magnesium oxide, calcium carbonate, magnesium aluminum silicate and other mineral bases.

4. The pharmaceutical formulation of claim 1, wherein said formulation comprises Phenytoin sodium or a pharmaceutically acceptable derivative thereof in extended or modified release form.

5. The pharmaceutical formulation of claim 1, wherein the Phenytoin sodium comprises about 5% to about 90% (weight percent) of the pharmaceutical formulation.

6. The pharmaceutical formulation of claim 1, wherein the amount of said phenytoin sodium comprises about 30 mg to about 300 mg per pharmaceutical formulation.

7. The pharmaceutical formulation of claim 1, wherein the formulation is an oral solid dosage form.

8. The pharmaceutical formulation of claim 7, wherein the formulation is a capsule, granule, granules in a capsule, tablet, pill, a caplet, pellets, spheroids or combinations thereof.

9. The pharmaceutical formulation of claim 7, wherein the formulation is a capsule or granules in a capsule.

10. The pharmaceutical formulation of claim 1, wherein the capsule or granules in a capsule comprises extended release phenytoin sodium or a pharmaceutically acceptable derivative thereof and at least one or more pharmaceutically acceptable excipient.

11. The pharmaceutical formulation of claim 1, wherein the formulation is prepared by using a wet granulation process comprising, preparing the wet granulation wherein phenytoin sodium or a pharmaceutically acceptable derivative thereof and at least one pharmaceutically acceptable excipient are blended together and granulated with nonaqueous solvents, optionally using binders; the wet granulation is screened, the granules are dried, and the dry granules are screened.

12. The pharmaceutical formulation of claim 1, wherein the formulation is prepared by using a wet granulation process comprising, preparing the wet granulation wherein phenytoin sodium or a pharmaceutically acceptable derivative thereof and at least one pharmaceutically acceptable excipient are blended together and granulated with nonaqueous solvents, optionally using binders; the wet granulation is screened, the granules are dried, and the dry granulation is combined with a lubricant.

13. The pharmaceutical formulation in solid unit dosage form of claim 1, wherein the formulation is prepared by a process comprising blending together phenytoin sodium or a pharmaceutically acceptable derivative thereof and at least one or more pharmaceutically acceptable excipient such as hydroxypropyl methyl cellulose and magnesium oxide; granulating with nonaqueous solvents; drying the granules; adding colloidal silicon dioxide and talc with blending; and adding magnesium stearate with blending.

14. The pharmaceutical formulation in solid unit dosage form of claim 13, wherein the final granules are filled into capsules using suitable capsule filling machine.

15. The pharmaceutical composition comprising phenytoin sodium or a pharmaceutically acceptable derivative thereof and at least one or more pharmaceutically acceptable excipient, wherein the composition has the characteristic Raman Shift between 1600 and 1700 cm⁻¹.

16. The pharmaceutical formulation of claim 1, wherein mean plasma concentrations of Phenytoin sodium or a pharmaceutically acceptable derivative thereof, following single dose oral administration of extended or modified release formulation, are comparable to the mean plasma concentrations of phenytoin sodium, following the administration of pharmaceutically acceptable dosage of the reference Kapseals® extended release Phenytoin formulation.

17. The pharmaceutical formulation comprising capsules or granules in a capsule, wherein said granules are prepared from blending phenytoin sodium or a pharmaceutically acceptable derivative thereof and at least one or more suitable pharmaceutically acceptable excipients and granulating the blend with one or more non-aqueous solvents, optionally using binders; and screening the granules after drying them and blending the same with glidants and lubricants and filled in the capsules.

18. The pharmaceutical formulation of claim 17, wherein the extended or modified release core comprises phenytoin sodium or a pharmaceutically acceptable derivative thereof and at least one or more suitable pharmaceutically acceptable excipient manufactured by using suitable granulation technique.

19. The pharmaceutical formulation of claim 17, wherein the granules are manufactured by using non-aqueous granulation technique.

20. The pharmaceutical formulation of claim 19, wherein said composition is in the form of capsules, granules in capsules, tablets in capsules, granules, slugs, beads, powder or combinations thereof.

21. The pharmaceutical formulation of claim 20, wherein said composition is capsule or granules in capsule.

22. The pharmaceutical composition of claim 17, wherein the said composition comprising phenytoin sodium or a pharmaceutically acceptable derivative thereof and at least one or more pharmaceutically acceptable excipient has the characteristic Raman shift between 1600 and 1700 cm⁻¹.

23. The pharmaceutical composition of claim 17, wherein the said pharmaceutically acceptable excipient is selected from the group consisting of microcrystalline cellulose, dibasic calcium phosphate, hydroxypropyl methyl cellulose, powdered cellulose, carboxymethyl cellulose, hydroxyethylcellulose and other cellulose derivatives, magnesium oxide, calcium carbonate, magnesium aluminum silicate and other mineral bases.

24. The pharmaceutical formulation of claim 17, wherein the mean plasma concentration of phenytoin sodium or a pharmaceutically acceptable derivative thereof, following single dose oral administration of extended or modified release formulation, are comparable to the mean plasma concentrations of phenytoin sodium, following the administration of pharmaceutically acceptable dosage of the reference Kapseals® extended release phenytoin formulation.

25. A process for manufacture of extended or modified release formulation of Phenytoin sodium or a pharmaceutically acceptable derivative thereof, the process comprising the steps of: (a) mixing phenytoin sodium or a pharmaceutically acceptable derivative thereof and at least one or more suitable pharmaceutically acceptable excipients (b) blending the mixture using a suitable blender (c) granulating the resulting mixture using organic solvent or a mixture of solvents optionally with binder (d) adding extragranular excipients such as disintegrants, glidants, lubricants and blending the resulting mixture in a suitable blender (e) filling the resulting granules into capsules.

26. A pharmaceutical formulation in solid dosage form prepared by process of claim 25, wherein the said formulation comprises blending together phenytoin sodium or its pharmaceutically acceptable derivative thereof and at least one or more pharmaceutically acceptable excipient such as hydroxypropyl methyl cellulose and magnesium oxide; granulating with nonaqueous solvents; drying the granules; adding colloidal silicon dioxide and talc with blending; and adding magnesium stearate with blending.

27. A pharmaceutical formulation in solid dosage form prepared by process of claim 26, wherein the final granules are filled in capsules using suitable capsule filling machine.

28. A pharmaceutical formulation in solid dosage form prepared by process of claim 25, wherein the formulation comprises phenytoin sodium or a pharmaceutically acceptable derivative thereof and at least one or more pharmaceutically acceptable excipient, and the said composition/blend has the characteristic Raman shift between 1600 to 1700 cm⁻.

29. A process of claim 25, wherein the said suitable pharmaceutically acceptable excipients are selected from the group consisting of microcrystalline cellulose, dibasic calcium phosphate, hydroxypropyl methyl cellulose, powdered cellulose, carboxymethyl cellulose, hydroxyethylcellulose and other cellulose derivatives, magnesium oxide, calcium carbonate, magnesium aluminum silicate and other mineral bases.

30. The pharmaceutical formulation of claim 1, wherein said drug is present in an amount sufficient to extend the in-vitro dissolution rate of the formulation over about a two hour time period.

31. A method of treating epilepsy and related disorders in a subject in need of treatment, which method comprises administering to the subject a pharmaceutical formulation of claim 1.

32. An extended or modified release formulation of claim 1, that provides a peak blood plasma level of phenytoin sodium within about 2 hours, said formulation containing phenytoin sodium or its pharmaceutically equivalent salts as the active ingredient.
